(19) United States
(12) Patent Application Publication
(10) Pub. No.: US 2008/0118411 A1
(43) Pub. Date: May 22, 2008

(54) OZONE DEODORIZING AND DISINFECTING PORTABLE CONTAINERS

(76) Inventor: Ralph D'Arinzo, Stamford, CT (US)

Correspondence Address: BAKER & MCKENZIE LLP, 1114 AVENUE OF THE AMERICAS, NEW YORK, NY, 10036, US

(21) Appl. No.: 11/562,136

(22) Filed: Nov. 21, 2006

(51) Int. Cl. B01J 19/00

(52) U.S. Cl. 422/186.09

(57) ABSTRACT

A portable storage apparatus for disinfecting, deodorizing and drying objects. The device for the apparatus comprising: a bag having at least one interior chamber formed by at least one outer wall for containing objects therein, ozone generator means for creating ozone, air distribution means for the movement and circulation of ozonated air within, throughout and out of the at least one interior chamber. The ozone generation means and air circulation means are located within or in direct circulatory contact with the one interior chamber of the bag to force ozonated air into and throughout the one interior chamber of the bag, and means on the outer wall permitting expultion of air from within the interior chamber of the bag to the environment outside the bag. Resulting in the disinfection, deodorization and drying of objects placed within the apparatus through contact with the ozone and air distributed therein.

### BACKGROUND OF THE INVENTION

The present invention relates to methods, apparatus and systems for deodorizing and disinfecting the contents of portable containers. More particularly, the present invention provides methods, apparatus and systems for producing high concentrations of ozone in portable containers for deodorizing and disinfecting the contents thereof.

Damp garments, sports and athletic equipment, clothing, boots and shoes have previously been dried in storage areas having natural air circulation. Garments and sports equipment that retain water and perspiration will mold, mildew and have unpleasant odors without adequate and timely removal of the moisture from the equipment.

U.S. Pat. No. 5,369,893 discloses several self-contained dryers for accommodating articles, such as clothing, sporting equipment and shoes. The dryers have walls that cause heated air to re-circulate in the drying chambers to decrease the moisture content of the air and increase the drying energy efficiency. Blowers and fans associated with electric heaters operate to circulate heated air through the drying chambers.

The dryer shown in FIGS. 23 to 26 of U.S. Pat. No. 5,369,892 is a chest or foot locker structure having a rigid housing. Wheels and a handle attached to the housing are used to facilitate manual movement of the dryer. The top of the housing has a cover movable to an open position to allow access to the drying chamber. A motor driven fan located in an air mixing chamber within the housing draws air through a filter into the drying chamber. Air porous walls within the drying chamber allow air to flow from the drying chamber back to the air mixing chamber for re-circulation back into the drying chamber. Air also flows out of the drying chamber through a filter having activated charcoal to remove odors from the air discharged into the environment from the chamber. This dryer requires an internal motor and fan associated with an electric heater, and specialized porous walls. While this device effectively dries articles, it is complex and expensive to manufacture. Moreover, the device only treats the ambient odors and not their underlying cause.

While both devices described above provide bags or vessels for drying damp articles stored therein neither provide a solution to the chemicals, odors, bacteria, microorganisms and other contaminants left behind that produce unpleasant and unhealthy odors, and in some cases are dangerous to breath or to come in physical contact with. In many cases, unpleasant odors and other contaminants are formed from perspiration and living organisms, such as bacteria left behind after the article is worn or used.

Storage of articles prone to such perspiration and/or bacterial contamination make putrefaction and decay a continual odor source, a nuisance, and in some cases a danger to the individuals using the contents stored therein. The storage or transfer of items prone to such sources of odor and contaminants within containers, bags or other vessels, air-tight or otherwise, typically provide fertile grounds for breeding dangerous and infectious bacterial organisms.

There are many known means for masking odors. One such means of odor masking superimposes one malodor on another stronger odor, thereby creating a new and often unpleasant odor. The method is typically used in waste disposal areas and many users do not like the odors produced from the masking materials that are usually chemically derived. Moreover, this method only lasts as long as a continuous supply of the masking material is supplied, and requires placing chemicals, fogs, mists or sprays into the ambient air or onto the contents of the subject container.

Another means comprises using chemical reactions that provide a more lasting deodorizing effect. These generally are accomplished by using chemical concentrates in liquid, block and granular forms. These methods are used mostly because they are low in cost, but usually are pollutants themselves because of the chemical fumes and gases that they release.

Biological methods of combating odor, such as using enzymes and bacteria to breakdown odor causing matter, are not always effective. Enzymes, for example, are slow acting, limited in their use and versatility, and work only on a limited variety of malodorous substances and are slow acting.

Filters and electronic air cleaners are often used to clean air in large areas. These devices include air exchange filtration systems, or electrically charged fields and plates to collect and remove particle matter. Although often effective for larger areas (e.g., locker rooms, gymnasiums, etc.), they are costly to use and maintain. In addition, they fail to target odors at the source, relying instead on a broader air purification of the ambient atmosphere. In reality, such cleaners only treat a symptom while permitting the problem to grow.

As mentioned above, there is a need for a mode of disinfecting and deodorizing damp or wet articles of clothing (e.g., sports equipment, bullet proof vests, shoes, toys and the like), while in storage or transit that can provide a less costly, efficient and easily mobile means for drying the clothing and combating odor and the underlying contaminants. By way of example only, sports equipment used in connection with ice hockey, roller hockey, football, lacrosse and various equestrian events are stored and transported in light weight containers and bags. Participation in each of the aforementioned activities, and the use of the requisite equipment therefore, results in the participants' perspiration and the absorption by such equipment and clothing of the same. The equipment, saturated with perspiration, provides a fertile breeding ground for bacterial action and the formation of unpleasant and sometimes unhealthy contaminants while within these bags or containers.

“Ozonation” is one of many methods used for the purification of diverse substances. Ozonation is more effective than odor masking methods, because ozone not only can kill bacteria and destroy viruses, but can also oxidize organics such as waste, and inorganics such as iron, magnesia, organically bound heavy metals, cyanides, sulfides and nitrates. Ozone can be used to remove odors (perspiration, urine, smoke, gases, water damage, pet and other domesticated animal odors, etc.), and also to destroy bacteria, mold, viruses, mildew and germs.

Ozone is an unstable gas. Oxygen, which is usually bi-atomic, becomes ozone after the addition of a third oxygen atom. The resulting molecule is very unstable and has a half-life of only minutes. Once ozone has lost its third atom, and done its job, it converts back to oxygen. In most cases, ozone has a half-life of less than twenty (20) minutes. In view of this fact, ozone cannot be stored for use at a later time. Rather, it must be produced at the point of use.

Ozone can be produced artificially through two different processes. The first is with ultra violet light in the 185 nanometer wavelength. While this procedure produces ozone, its production is not in high concentrations or strengths. The advantage of the ultra violet systems is that it is pretty much maintenance free for a period of up to 10,000 hours of use.

The second process of producing ozone is called corona discharge. This process takes a high voltage arc, usually 10,000 volts or higher, and passes it through a dielectric to generate an arc that will produce high concentrations of ozone.

Devices that produce ozone through electrolysis and employ ozone for deodorization or disinfecting are known in the art. Many of these devices, however, are not suited to particular applications, are of complex construction and comprise parts that are not readily available. Moreover, many of these devices for producing ozone are large, expensive, cumbersome and difficult to transport, as well as complicated enough to preclude their operation by unskilled individuals. It is not desirable that ozone be generated over a wide area, but rather directed specifically to the object to be deodorized or disinfected.

U.S. Pat. No. 6,134,806 describes an apparatus for deodorizing the contents of a portable sports equipment bag. The prior art device depicted in FIG. 1 comprises a fan or blower X connected by multiple hoses and pipes to an ozone generator Z which in turn is connected by hose Y to the sports equipment bag W. This device like so many of these devices, is clumsy and difficult to transport and handle. Indeed, all three of the components are bulky and necessary for the device to function. Moreover, assembly and disassembly of the device by the user is required each time it is transported and used.

### SUMMARY OF THE INVENTION

The present invention comprises portable bags, vessels and containers having at least one internal chamber for storing articles, such as clothing, shoes, boots, ice skates, ski boots, sporting equipment and other objects. A lightweight air distributor located or housed within or adjacent to the chamber directs a steady stream of pressurized air into the chamber to dry the articles stored in the chamber. An ozone generator associated with the air distributor that moves ozone into the chamber with the pressurized air. The ozone functions as an oxidant and germicide on the equipment and/or contents of the bag. The bag is either constructed from porous materials or has pressure release valves or vents mounted on the container or vessel walls to maintain a desired elevated air pressure within the chamber while allowing air to flow out of the bag or container.

The bags, containers and vessels of the present invention are each equipped with at least one air distributor and ozone generator. The present invention provides effective means for drying damp articles stored within the bag or container without the need for bulky, complex and expensive designs and components, while also ensuring the removal and neutralization of malodors and unhealthy contaminants from the chamber and its contents.

The blower can be equipped with an electric heater to heat the air discharged by the blower into the chamber. The air is naturally distributed throughout the bag as a result of pressure caused by the steady stream produced by the air distributor. This increases drying efficiency and reduces damp sections of the articles. The blower may also have a timer or sensor that will stop further ozone production and air distribution when the desired result has been attained.

In one embodiment of the present invention the bag can be made from porous materials such as canvas, vinyl, plastic or fabric materials that allow it to be folded for storage and expanded for accommodating articles. The bag can have a longitudinal or vertical orientation. The bag may also include wheel and a handle at opposing ends to facilitate movement of the bag.

The bag may have a single or multiple compartments. The bag may also include side pockets for storing specific articles, such as shoes, ice skates or gloves. The ozone generator supplies ozonated air in the chamber(s) containing articles requiring treatment. Ozonated air may also be introduced into the side pockets if such design is desirable. In either case, the ozonated air may be introduced into the chambers or side pockets through either porous materials of construction or vents mounted on the walls between the main chamber(s) and the side pockets.

The bag will include means for opening and closing the bag and its various compartments and pockets as the case may be. Such means may include one or more zippers or releasable fasteners (e.g., Velcro, buttons or the like) that ensure easy access into the bag's chamber(s) and pocket(s), as the case may be, to facilitate the placement and removal of the articles into and from the bag.

In a second embodiment of the present invention, the device may be constructed using rigid containers and vessels. Such containers and vessels, may be constructed from various substances including plastic, urethane, rubber, metal or vinyl. These containers may take on various shapes and configurations, and may have a single or multiple chambers, depending upon their intended use. In the case of containers with multiple chambers, such chambers may or may not receive ozonated air and may or may not contain vents or the like, again, depending upon their desired use.

These containers may be air tight in certain circumstances depending upon the container's intended application. However, it is not necessary to ensure the successful removal and neutralization of moisture and contaminants by the present invention. Examples of existing airtight containers that may be easily modified for use with the present invention are commercially available from, among others, RUBBERMAID® and TUPPERWARE®.

Air tight vessels and containers will require vents or pressure release valves to maintain a steady pressure and steady flow of ozonated air throughout the container. Such vents and valves may be formed as part of the containers walls or mounted on the container walls. In the case of containers with multiple chambers vents or valves may be formed as part of or mounted on the container walls to permit ozonated air to flow through out the various chambers and out of the container, resulting in the circulation of the ozonated air and the removal of odors and particulates from the articles stored therein. In certain circumstances it may be desirable to have filters associated with the vents and valves to clean or scrub the air exiting the container.

The ozonated air circulated through out the bag's or container's compartments picks up the moisture, foreign matter and odors from the articles placed in the bag chamber. The ozone neutralizes the odors, bacteria, molds and/or fungus associated with the stored items. The foreign matter (odors and destroyed bacteria or the like), neutralized by the ozone are carried by the air circulated through the compartments and is expelled from on the bag or container through the valves, vents or pores in the materials of construction. Pressure valves, vents or pore density may be used to maintain the desired air pressure within the bag or container.

### DESCRIPTION OF THE DRAWINGS

FIG. 1 is a perspective view of a prior art device comprising a bag, air blower and ozone generator connected by hoses.

FIG. 2 is a perspective view of a multi-chamber travel and sport bag equipped with the ozonated air distribution system of the present invention.

FIG. 3. is a perspective view of the sports and travel bag illustrated in FIG. 2 having a cutaway section providing a view inside the bag's chambers and the ozonated air generating unit housed therein.

FIG. 4 is a sectional view taken along line A-A of FIG. 2.

FIG. 5 is a perspective view of a multi-chamber container equipped with the air ozonation system of the invention.

FIG. 6 is a perspective view of the container illustrated in FIG. 5 with a cutaway section providing a view of the container's inner chambers and to ozone generating unit housed therein.

FIG. 7 is a perspective view of a foot locker with a hinged top in its open position exposing the contents therein that is equipped with the ozonated air distribution system of the present invention.

FIG. 8 is a perspective view of a shoe storage bag with a flap top in its open position exposing the contents therein that is equipped with the ozonated air distribution system of the present invention.

FIG. 9 is a perspective view of a garment bag with a zipper top in its closed position that is equipped with the ozonated air distribution system of the present invention.

FIG. 10 is a perspective view of a knap-sack with a buttoned flap top in its closed position that is equipped with the ozonated air distribution system of the present invention.

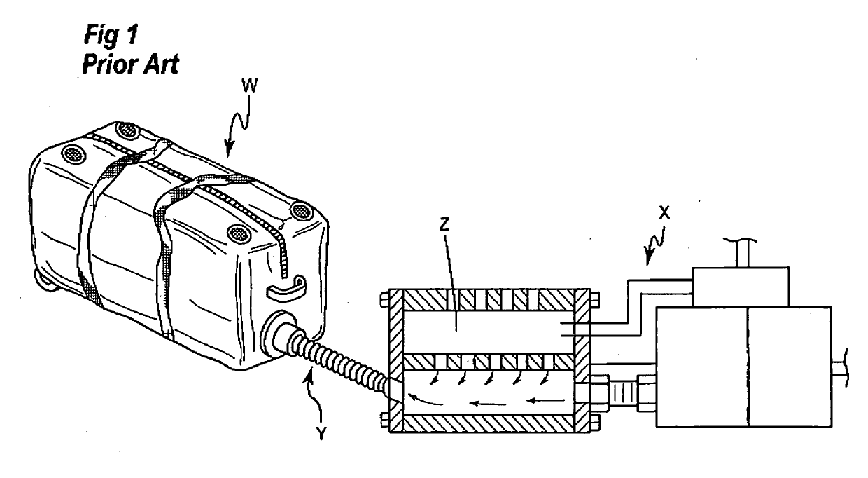

### DETAILED DESCRIPTION OF PREFERRED EMBODIMENTS

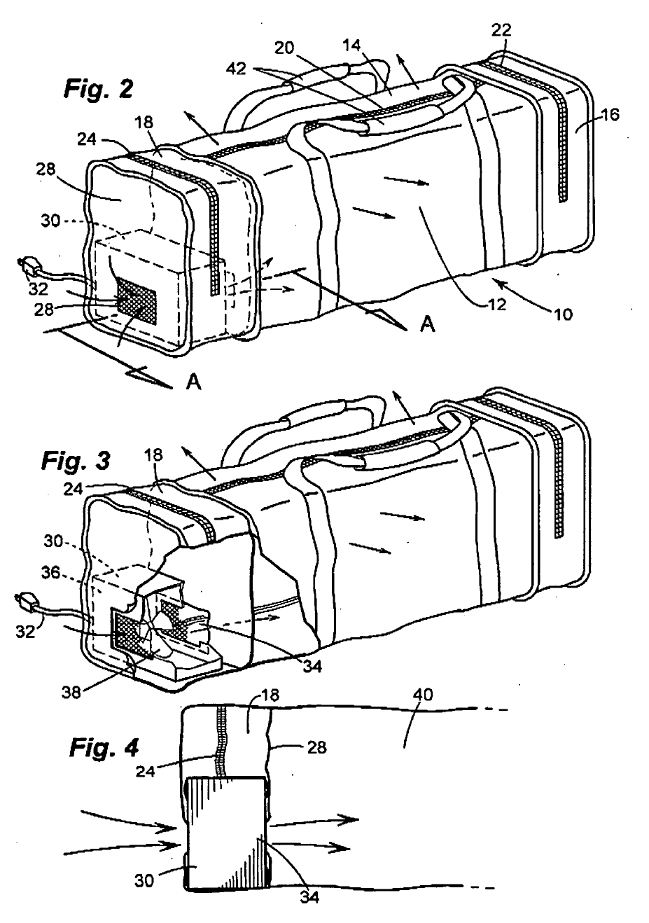

The travel and sports bag 10, shown in FIG. 2, has a porous outer skin 12 constructed from a flexible canvas, plastic, vinyl or cloth material. Bag 10 may be used to store and carry, among other things, sports equipment, such as hockey, lacrosse, soccer, football and equestrian equipment. The equipment stored in bag 10 is typically placed in the bag after use in a moist, damp and usually wet condition as a result of the athlete's body sweat, the plain conditions (e.g., weather) and the plain surface (ice in the case of ice hockey, mud and equine perspiration in the case of equestrian events.

While bag 10 may be constructed with only a single inner compartment, it is preferred that the bag contain multiple compartments so that articles may be organized and stored more easily. As depicted in FIGS. 2 and 3, bag 10 has 3 compartments, 14, 16 and 18. Zippers 20, 22 and 24 facilitate easy access for the placing and removal of items stored in compartments 14, 16 and 18 respectively. While FIGS. 2 and 3 depict the use of zippers, there are several known means for the opening and closing of the bag and its various compartments (e.g., Velcro, hooks, buttons) from below. Easy transportation of the bag may be accomplished through various means including, for example, straps 42 for carrying the bag or a handle on one of the longitudinal ends of the bag with wheels or rollers (not depicted) on the opposing end of the bag for towing.

As indicated by the arrows, ozonated air is moved through bag 10. More precisely, air is sucked through air intake vent 26 into compartment 18 which may be supported by inner wall 28. This wall 28 may be constructed in a variety of ways including a rigid sheet fitted to cover the entire inner surface of compartment 18 constructed from cardboard, plastic or metal. Support for compartment 18 may also be accomplished through the use of ribs or slats inserted into or affixed to the wall. While not essential, such support is preferred.

Ozonated air generator unit 30 is housed within compartment 18 of bag 10. Preferably, unit 30 is electrically powered, relying upon either power cord 32 or batteries (not depicted) or a rechargeable cell (not depicted). While there are several units currently available that would work with the present invention, the unit depicted and disclosed with the present invention is commercially available through International Ozone Technologies Group Inc., under the brand name, TOTAL ZONE.

As depicted in FIG. 3, unit 30 is encased in a plastic or metal casing 36, which houses at least one air blower including a fan or propeller 38 which is driven by a motor (not depicted) and an ozone generator (not depicted). Air sucked in through air intake vent 26 is ozonated by ozone generator unit 30 The ozonated air is pushed through distribution vent 34 into inner chamber 40. The ozonated air continues to circulate throughout the inner chamber 40 and the contents stored therein where it functions as an oxidant and germicide.

The ozone generator is a conventional ozone producing module which produces ozone by passing through a corona discharge between two parallel or concentric electrodes separated by a dielectric. The oxygen in the air is broken down and charged oxygen atoms recombine to form ozone molecules. Unit 30 may additionally feature at least one electric timer, a heating unit, or a pressure sensor.

As the pressure within inner chamber 40 increases, flow is maintained through the discharge of air and particulate matter separated from the items stored within bag 10 by the ozone through the porous outer skin 12. In an alternate embodiment, bay 10 may incorporate pressure valves, vents or vented filters to ensure the flow of the air out of the bag. This maintains a constant and steady stream of ozonated air through the bag.

It is important to note that while not necessary for the invention to function, it, may be desirable depending upon the bag's application to include filters with the vents valves. The air expelled from the bag will remove micro particles of bacteria and dirt destroyed and removed from the articles by the ozone. Most if not all of the ozone introduced into the inner chamber and the articles stored therein will have converted back to oxygen, having reacted in its normal course. Bag 10 may additionally include straps 42 or the like for easy transport.

In a second preferred embodiment of the present invention, container 50 is constructed from a non-porous rigid material such as plastic. While it is not necessary for the invention to function, it may be preferred depending upon the application, that contain 50 be air-tight. Containers of similar construction are regularly commercially available through manufacturers such as RUBBERMAID®. Container 50 may be used for the storage of various articles including, for example, sports equipment or toys. These articles, through ordinary use, often collect moisture and bodily fluids (e.g. saliva, sweat), which ultimately can result in the growth of bacteria and mildew, and the development of an unpleasant odor. The present invention provides effective means for drying and decontaminating moist and damp articles stored within the container without the need for bulky, complex and expensive equipment or the use of dangerous and oftentimes unpleasant chemicals. Easy transportation of the container bag may be accomplished through various means including, for example, the inclusion of handles either attached to, or molded into the container or a handle on one of the longitudinal ends of the container with wheels or rollers (not depicted) on the opposing end of the bag for towing.

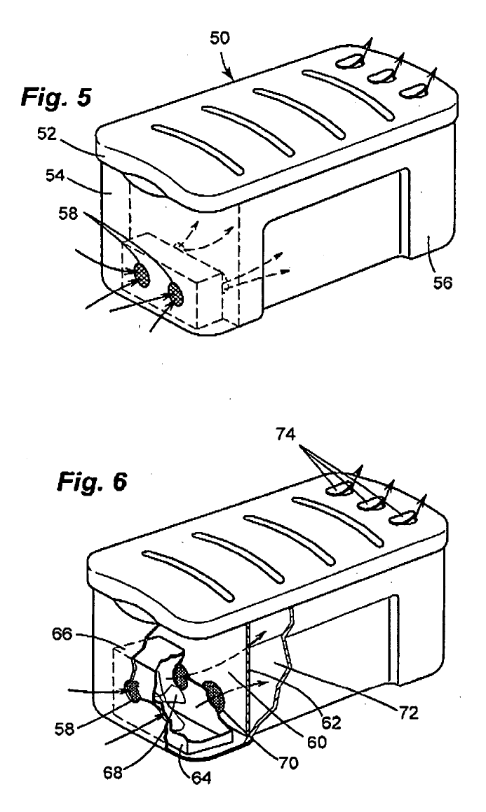

As depicted in FIGS. 5 and 6, container 50 has a two-part construction comprising container lid 52 and container base 54, which cooperate to form an air-tight (as depicted) or nearly air-tight seal (depending upon the application) between the two. Container lid 59 should fit firmly, but be easily removable and replaceable to facilitate easy access to the container's inner components for removing or placing objects within the container. Outer walls 56 of container 50 may take on several shapes depending upon the containers' intended use.

Like the prior embodiment, air is drawn in through an air intake vent or valve 58 (depending again upon the desired use of the container). While the container may have only a single compartment within formed by the walls 56 of base 54 in cooperation with lid 52, the present embodiment utilizes two compartments. In other applications, it is likely that several additional compartments could be created depending again upon the application for which the container is designed. As illustrated in FIG. 6, container 50 has two compartments: a first compartment 60 which houses the ozonated air generator unit, and a second inner storage compartment or chamber 72.

Ozonated air generator unit 64 is removably attached to inner wall 62. Unit 64 while securely fastened while in use may be easily removed for service or repair without the use of tools.

As depicted in FIG. 6, unit 64 is encased in a plastic or metal casing 66, which houses at least one air blower including a fan or propeller 68 which is driven by a motor (not depicted) and an ozone generator (not depicted). Air sucked in through air intake vents 58 is ozonated by ozone generator unit 64 The ozonated air is pushed through distribution vents 70 into inner compartment 72. The ozonated air continues to circulate throughout inner compartment 72 and the contents stored therein where it functions as an oxidant and germicide.

The ozone generator is a conventional ozone producing module which produces ozone by passing through a corona discharge between two parallel or concentric electrodes separated by a dielectric. The oxygen in the air is broken down and charged oxygen atoms recombine to form ozone molecules. Unit 64 may additionally feature at least one electric timer, a heating unit, or a pressure sensor.

Ozonated air distribution vent or valve 70 is located on inner wall 62 in immediate proximity with ozonated air generator unit 64. As presently depicted, there are two vents 70, however, this number can vary again depending upon the application for which the container is designed. It is important to note that if additional compartments are desired, vents or valves of the nature of those depicted here would need to be placed on the walls leading into and out of such compartments in order to ensure the constant and steady flow of ozonated air throughout the container. If, however, it is desired that a portion of the container provide an ozone-free environment, such compartment would require construction of walls without vents or valves, or alternatively with vents or valves that may be shut air-tight when desired.

In order to ensure a constant stream of air through the container while maintaining a desired pressure therein, the container will be equipped with exhaust vents or valves 74 that will be positioned on its exterior. In this case, exhaust valves 74 consist of flaps designed to permit air to escape from the container when a desired pressure has been reached. Although not depicted, such exhaust valves 74 may contain filters (activated charcoal, fiberglass, paper) which could be used to scrub air as it exits the container. Most if not all of the ozone introduced into the inner compartment and the articles stored therein will have converted back to oxygen, having reacted in its normal course. Container 50 may additionally include straps 42 or the like for easy transport.

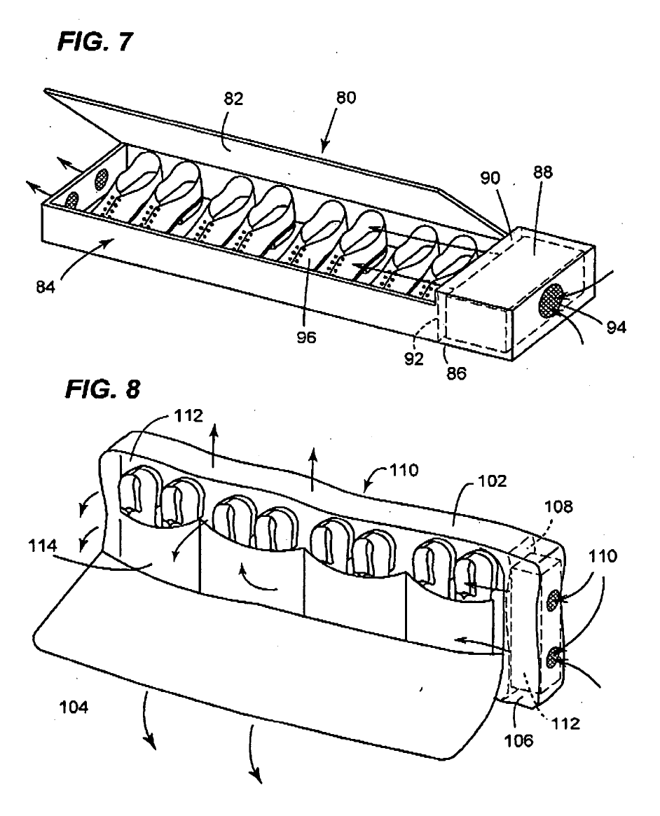

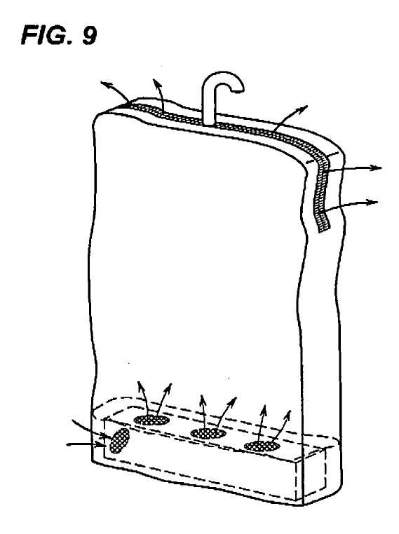

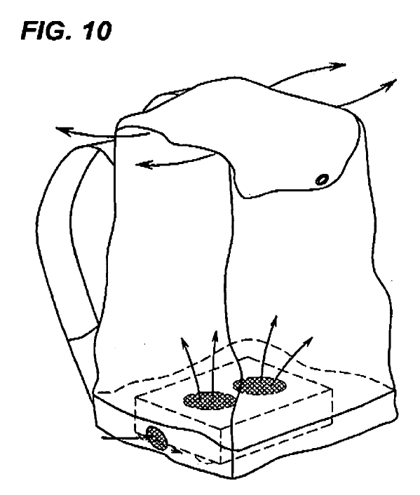

The ozonated air distribution system of the present invention may be adapted for use with bags and containers of various configurations commonly used for storage and/or transportation of garments, sports and military equipment, toys or the like. FIGS. 7, 8, 9 and 10 demonstrate the systems versatility.

While there has been shown and described certain preferred embodiments of the ozonated air distribution system for bags and containers of the present invention, it is understood that changes in the structures, materials of construction, and arrangement of the system's components can be made by a person skilled in the art without departing from the invention. The invention is defined in the following claims.

What is claimed is:

1. A portable storage apparatus for disinfecting, deodorizing and drying objects comprising: a bag having at least one interior chamber formed by at least one outer wall for containing objects therein, ozone generator means for creating ozone, air distribution means for the movement and circulation of ozonated air within, thoughout and out of the at least one interior chamber, the ozone generation means and air circulation means being located within or in direct circulatory contact with the at least one interior chamber of the bag to force ozonated air into and throughout the at least one interior chamber of the bag, and means on the at least one outer wall permitting expultion of air from within the at least one interior chamber of the bag to the environment outside the bag, whereby objects placed in the at least one interior chamber are disinfected, deodorized and dried through contact with the ozone and air distributed therein.

2. The apparatus of claim 1, wherein the bag has at least one interior wall which is shaped to cooperate with at least one outer wall to form at least two inner chambers, the ozone generator means and the air distributor means housed in the first chamber and the at least second chamber housing objects placed within the bag, whereby ozonated air is forced by means for moving ozonated air from the first chamber into and throughout the second chamber of the bag causing the objects housed within the bag to be disinfected, deodorized and dried through contact with the ozone and air distributed therein.

3. The apparatus of claim 1, wherein the bag has closure means secured to the bag operable to provide access to the at least one interior chamber, whereby objects may be stored within or removed from the at least one interior chamber.

4. The apparatus of claim 1 wherein: the means for the movement and circulation of ozonated air within, thoughout and out of the at least one interior chamber air is at least one of the following group: fan, air blower, valve, flap, aperture in the interior wall, or an inner wall having a porous construction.

5. The apparatus of claim 1 wherein: the air distributor means has a wall facing the interior chamber of the bag and means comprising at least one hole in the wall to allow ozonated air to flow from the first interior chamber to the second interior chamber of the bag to dry objects in the bag.

6. The apparatus of claim 1 wherein: the bag includes pocket means having pocket chambers for accommodating objects to be dried, and means including apertures in the pocket chambers for carrying ozonated air from the chamber means into the pocket chambers to dry objects located within the pocket chambers.

7. The apparatus of claim 1 including: wheel means secured to one end of the bag for supporting the bag on a surface, and handle means secured to the other end of the bag to facilitate transport of the bag on said surface.

8. The apparatus of claim 1 including: straps secured to the bag providing handles to manually carry the bag containing the objects.

9. An apparatus for drying objects comprising: a container having an interior chamber for accommodating objects, a device for generating ozone, control means connected to the device for regulating the duration of operation of the device for regulating the duration of operation of the device to limit the amount of ozone generated by the device, air distributor means located within the interior chamber of the container, said air distributor means having chamber means for accommodating air and ozone, first means to allow air from a supply of air and ozone from the device to flow into the chamber means, second means to allow air and ozone to flow from the chamber means into the interior chamber of the container to dry objects in the interior chamber and destroy bacteria, molds and fungus in the interior chamber, and means to allow air to flow from the interior chamber of the container to the environment adjacent the container.

10. The apparatus of claim 9 wherein: the container has a bottom wall, said air distributor means being located on said bottom wall with said second means directed away from the bottom wall to direct air and ozone upward into the interior chamber of the container and closure means secured to the container operable to provide access to the interior chamber of the container.

11. The apparatus of claim 9 wherein: the container includes pocket means having pocket chambers for accommodating objects to be dried, said second means including tubular means for carrying air and ozone from the chamber means into the pocket chambers to dry objects located within the pocket chambers and destroy bacteria, molds and fungus in the pocket chambers.

12. The apparatus of claim 9 including: wheel means secured to one end of the bag for supporting the bag on a surface, and handle means secured to the other end of the bag to facilitate transport of the bag on said surface.

13. The apparatus of claim 9 wherein: the means to allow ozone and air to flow from the interior chamber of the bag includes filter means mounted on the container to remove odors from the air flowing through the filter means to the environment adjacent the container.

14. The apparatus of claim 13 wherein: the filter means includes charcoal means and porous material for removing odors, particulates and foreign materials from the air flowing through the charcoal means and porous material.

15. A method of removing moisture and odors from objects and storing the objects in an apparatus having an enclosed interior chamber and an air distributor having chamber means for accommodating air and providing passages to allow air to flow from the chamber means into the interior chamber of the apparatus comprising:
a. confining the objects within the enclosed interior chamber of the apparatus,
b. generating ozone,
c. controlling the duration of generating ozone,
d. introducing air and ozone into the chamber means of the air distributor,
e. dispensing air and ozone from the air distributor into the interior chamber through the passages of the air distributor to remove moisture and odors from the objects located in the chamber, and destroy bacteria, molds and fungus in the chamber of the apparatus and objects in the chamber,
f. exhausting air from the chamber into the environment,
g. filtering the air exhausting from the interior chamber to remove foreign matter from the air exhausted into the environment,
h. storing the dried objects in the interior chamber of the apparatus, and
i. transporting the apparatus with the stored dried objects to selected location.

16. The method of claim 15 including: heating the air introduced into the chamber means of the air distributor.

17. The method of claim 15 including: storing an object in a second chamber in the bag separated from the enclosed interior chamber, and directing air and ozone from the passage of the air distributor into the second chamber to remove moisture and odors from the object in the second chamber, and exhausting air from the second chamber.

18. The method of claim 15 including: filtering the air exhausting from the second chamber to remove odors from the air exhausting from the second chamber.
